(19) United States
(12) Patent Application Publication
(10) Pub. No.: US 2020/0008825 A1
(43) Pub. Date: Jan. 9, 2020

(54) SUSTAINED INTRATONSILLAR DRUG DELIVERY WITH NEEDLE ARRAY

(71) Applicants: SOUTHERN RESEARCH INSTITUTE, Birmingham, AL (US); THE UAB RESEARCH FOUNDATION, Birmingham, AL (US)

(72) Inventors: DO-YEON CHO, Homewood, AL (US); DONG-JIN LIM, Birmingham, AL (US); BRADFORD A. WOODWORTH, Mt. Brook, AL (US)

(21) Appl. No.: 16/480,910

(22) PCT Filed: Jan. 26, 2018
(86) PCT No.: PCT/US2018/015563

§ 371 Date: Jul. 25, 2019

Related U.S. Application Data
(60) Provisional application No. 62/451,173, filed on Jan. 27, 2017.

(51) Int. Cl. A61B 17/24; A61M 37/00

(52) U.S. Cl. CPC A61B 17/24; A61M 37/0015; A61M 2037/0023; A61M 2210/065; A61M 2037/0053; A61M 2037/0061; A61M 2037/0046

(57) ABSTRACT

Various embodiments for sustained drug delivery using a needle array are disclosed. A medical instrument and a method associated therewith are described for delivering drugs into tonsillar and other tissue as a way of treating tonsillar hypertrophy or inflammation, as an alternative to tonsillectomy and adenoidectomy (T&A) procedures. In some embodiments, a tonsil-accessible medical instrument is described as including a needle head having needles, or microneedles, disposed therein and capable of introducing a compound, such as a steroid, into tissue. The medical instruments enables for the delivery of various compounds, such as pharmaceuticals, for controlled and sustained release.

### CROSS-REFERENCE TO RELATED APPLICATION

This application claims the benefit of and priority to U.S. Provisional Patent Application No. 62/451,173, entitled “SUSTAINED INTRATONSILLAR DRUG DELIVERY WITH NEEDLE ARRAY,” filed on Jan. 27, 2017, the contents of which being incorporated by reference in their entirety herein.

### BACKGROUND

Tonsillectomy and adenoidectomy (T&A) are common and often-frequent surgeries carried out in children and adults, often to treat obstructive sleep apnea with tonsillar hypertrophy and chronic tonsillitis. Although T&A has many benefits, it is an invasive surgical procedure where several significant complications can occur during or after the procedure, such as pain, delayed feeding ability, dehydration, postoperative hemorrhaging, and postoperative airway compromise. In some instances, serious complications such as death have been known to occur.

### BRIEF DESCRIPTION OF THE INVENTION

Various embodiments for sustained drug delivery using a needle array are disclosed. In various embodiments, a medical instrument and a method of use associated therewith are described for delivering drugs into the surface and core of the tonsils as a way of treating tonsillar hypertrophy and inflammation, which may avoid complications from surgical removal of tonsils. In some embodiments, a tonsil-accessible medical instrument is described as including a detachable needle coupled to a central member and handle portion, where the needle head has needles, or microneedles, disposed therein. The medical instrument enables for the delivery of various compounds, such as steroids, antibiotics, or other pharmaceuticals, for controlled and sustained release.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIGS. 1-3 include examples of a first embodiment of a medical instrument having a needle head for introducing a compound into tissue of a subject according to various embodiments of the present disclosure.

FIG. 4 is an enhanced view of the needle head of FIGS. 1-3 according to various embodiments of the present disclosure.

FIG. 5 is an enhanced cross-section of the needle head of FIGS. 1-4 according to various embodiments of the present disclosure.

FIG. 6 is an example of a second embodiment of a medical instrument having a needle head for introducing a compound into tissue of a subject according to various embodiments of the present disclosure.

FIG. 7 is an enhanced cross-section of the needle head of FIG. 1 or FIG. 6 according to various embodiments of the present disclosure.

FIG. 8 is another enhanced cross-section of the needle head of FIG. 1 or FIG. 6 according to various embodiments of the present disclosure.

FIG. 9 is another enhanced cross-section of the needle head of FIG. 1 according to various embodiments of the present disclosure.

FIG. 10 is a drawing of the medical instrument of FIG. 1 or FIG. 6 being used to introduce a compound into tonsillar tissue according to various embodiments of the present disclosure.

FIG. 11 includes a graph illustrating a percentage of cluster-of-differentiation-3 (CD3) T-cell co-receptors in a rabbit when treated with mometasone versus a control.

FIG. 12 includes a chart illustrating in vitro kinetics of a drug or compound released into tonsillar tissue.

FIG. 13 includes photographs of tonsillar tissue treated with a nanoparticle control and a mometasone nanoparticle according to various embodiments of the present disclosure.

FIG. 14 is a flowchart illustrating an example method for using the medical instrument of FIG. 1 or FIG. 6 to introduce a compound into tissue according to various embodiments of the present disclosure.

### DETAILED DESCRIPTION

The present disclosure relates to sustained and controlled intratonsillar drug delivery using a needle array. Various complications have known to occur as a result of a T&A procedure, including, but not limited to, vascular injury, subcutaneous emphysema, jugular vein thrombosis, atlantoaxial subluxation (Grisel syndrome), taste disorders, such as hypogeusia, ageusia, dysgeusia, phantogeusia, velopharyngeal insufficiency, and even death.

When treating tonsillar hypertrophy or tonsillitis—an infection and inflammation of the tonsils—nonsurgical treatment options are preferred to reduce the associated complications that affect the quality of life in a child or other human subject. Intranasal steroid sprays have been explored to treat adenoid hypertrophy. For instance, intranasal corticosteroids may significantly improve nasal obstruction symptoms in children with moderate to severe adenoidal hypertrophy, where the improvement includes a reduction in adenoid size. Moreover, cells treated with corticosteroids have been shown to result in marked dose-dependent reductions in proliferation rates, increased cellular apoptosis, and diminished cytokine release. However, the anatomical location of the tonsils and salivary washout often lead to the failure of nasal steroids. Additionally, salivary washout and the anatomical location of the oral cavity continues to challenge sustainable drug delivery.

According to various embodiments, focal tonsillar injections of corticosteroids and other compounds are described, for example, to reduce the size of palatine tonsils. Corticosteroid injections of fluticasone made on rabbits having enlarged palatine tonsils have significantly decreased the size of the tonsils when compared to those treated with saline. Additionally, multiple steroid injections have resulted in a signification reduction in tonsil size when compared to that of a single injection. In other words, a single injection of corticosteroids appears to be effective, but not as effective as multiple injections. Consequently, an efficient medical treatment for introducing a compound, such as fluticasone, dexamethasone, and budesonide, into the tonsils with a sustained release profile would be a non-invasive regime for treating tonsillitis and other infections associated with the tonsils.

When compared to oral delivery, the topical trans-dermal route has been regarded as a more efficient pathway to deliver drugs into the skin without a significant first-pass effect of the liver associated with oral forms. Trans-dermal delivery circumvents several problems, such as drastic pH changes, a deleterious presence of food and enzymes, variable transit times, and pulse entry. Similarly, the intra-tonsillar delivery can deliver drugs that are subjected to first-pass metabolism. Human skin has a relatively large surface area that can absorb drugs and other compounds; however, the human skin has a stratum corneum (SC) layer that prevents the diffusion of drugs through skin. The surface of the oropharynx—the middle part of the throat that includes the base of the tongue, the tonsils, the soft palate, and the walls of the pharynx—has a non-keratinized stratified squamous epithelium of about 40˜50 cell layers with a thickness of about 200˜400 μm. This results in about 4˜4000 times of higher permeability than the skin.

Accordingly, various embodiments for sustained intratonsillar drug delivery are disclosed, for example, using a needle array for introducing a compound into the tonsils or other suitable region of the oral or pharyngeal cavity. In one embodiment, a medical instrument includes a handle portion and a central member having a curvature that facilitates access to various regions of a cavity, such as the tonsils. A needle head may be coupled to a distal end of the handle portion or the central member where a needle array disposed on the needle head is configured with a compound that, when the needle array is introduced into tissue, causes a delivery of the compound into the subject. In some embodiments, the needle array may include an arrangement of needles or microneedles having a needle size suitable for introducing a compound into tissue, ideally without inducing pain or considerable reaction in a subject.

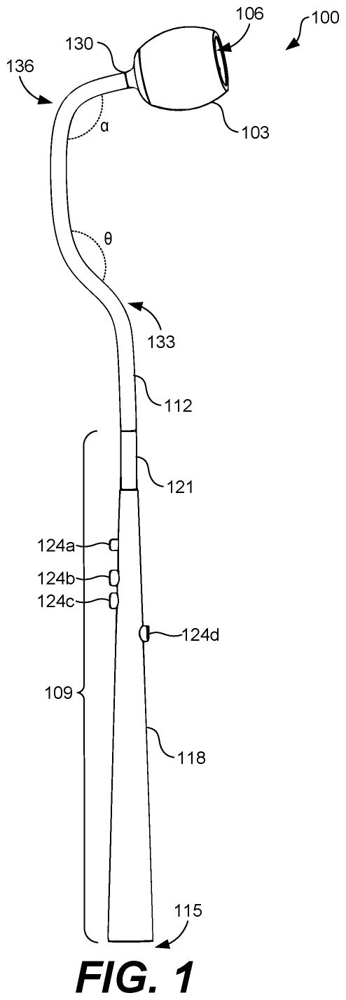

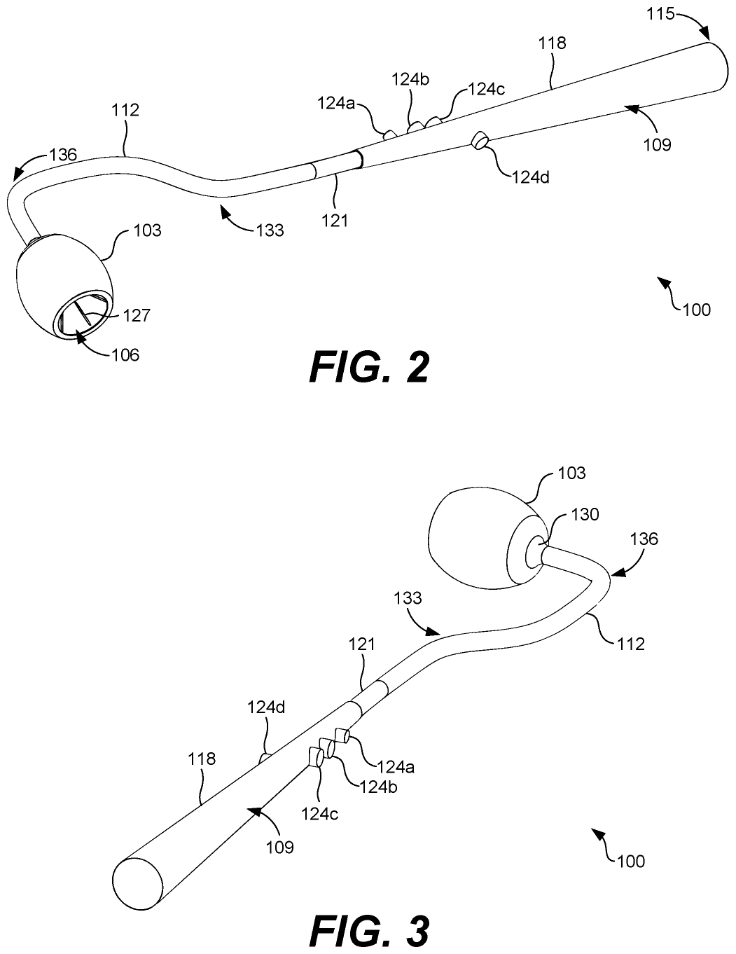

Referring now to FIGS. 1-4, an example embodiment of a medical instrument 100 for accessing the tonsils, or other appropriate region of the oral or pharyngeal cavity, is shown according to various embodiments. The medical instrument 100 may include a needle head 103 that includes a multitude of needles or microneedles disposed in a cavity 106 therein for introducing a compound into tissue of a subject, as will be discussed. The medical instrument 100 may further include, for example, a handle portion 109 adapted for an operator to hold or grip while using the medical instrument 100. The medical instrument 100 may also include a central member 112 that couples the handle portion 109 to the needle head 103. Additionally, the central member 112 may have a shape that facilitates placing needles disposed in the cavity 106 of the needle head 103 into tonsillar tissue or tissue in other regions of the body.

In some embodiments, the handle portion 109 and the central member 112 may include a hollow interior. To this end, in some embodiments, a terminal end 115 of the medical instrument 100 may be attached to a suction system to pull saliva and other material from an oral cavity through the cavity 106 of the needle head 103, through the interior of the central member 112 and the handle portion 109, and out through the terminal end 115 of the medical instrument 100. In other embodiments, the handle portion 109 can include a compound, for instance, in liquid form stored in the interior of the medical instrument 100 (or coupled to an external reservoir) that can be introduced into the subject through the needles disposed in the cavity 106 of the needle head 103.

The handle portion 109 may include a first elongated member 118 and a second elongated member 121 although, in other embodiments, other suitable number of members may be employed to form the handle portion 109. As shown in FIGS. 1-4, the first elongated member 118 may be longer than the second elongated member 121. Additionally, the first elongated member 118 and the second elongated member 121 may be cylindrical or rectangular, in some embodiments.

Further, the first elongated member 118 may be conical, where a base of the first elongated member 118 is wider and tapers upwards towards a top of the first elongated member 118. As may be appreciated, the first elongated member 118 may have another shape suitable for gripping by an operator. The handle portion 109 may further include one or more buttons 124a . . . 124d (collectively “buttons 124”) projecting from or flush with an exterior surface of the handle portion 109. In some embodiments, the buttons 124 can be configured to perform various functions associated with the medical instrument 100. For instance, one or more of the buttons 124 may be configured to: (a) toggle suction (e.g., to remove saliva or other substance from the oral cavity) when the medical instrument 100 is coupled to a suction system, (b) release the needle head 103 from the central member 112, (c) introduce drugs or other compounds from an interior of the medical instrument 100 or from an external reservoir, and (d) remove needles from the needle head 103 or other portion of the medical instrument 100.

The needle head 103 may be coupled to the handle portion 109, for example, at a distal end of the central member 112. The needle head 103 may include an arrangement of needles or needle array 127 associated or configured with a compound that causes a delivery of the compound into a subject when the needle array 127 is introduced into tissue. In some embodiments, the needle array 127 is formed by a multitude of microneedles (or a multitude of other sizes of needles) having a size suitable for introducing a compound into tissue. For example, the size of the needles in the needle array 127 may be chosen to deliver the compound efficiently based on the molecular size of drug, as well as to avoid introducing any complications, bleeding, or pain in a subject when the compound is administered.

Each of the needles in the needle array 127 may have a needle size of about 250 micrometers to 2,500 micrometers. In some embodiments, each of the needles in the needle array 127 may have a gauge of about 22 to 25, although other gauge needles may be employed. Further, the needle head 103 may be bowl-shaped. In other words, the needle head 103 may include a cavity 106 on a top and forward facing portion of a cylinder, where the cylinder may be circular or ovular. Through use of the handle portion 109, an operator may guide tonsillar or other tissue into the cavity 106 to introduce the needle array 127 into the tissue. In some embodiments, the needle head 103 may be detachably attached from the central member 112 at a coupling point 130. For instance, one of the buttons 124 may control the detachability of the needle head 103 from the central member 112. In another embodiments, the needle head 103 may be integrated with the central member 112.

As noted above, the central member 112 may be include a shape that facilitates placing the needle head 103 into an oral cavity or other portion of a body such that the needle array 127 can be easily introduced into tonsillar tissue or other tissue. To this end, in some embodiments, the central member 112 may include a C-shaped body. In this regard, the central member 112 may include a uniform body having a first bend 133 and a second bend 136. The first bend 133 may form a first angle (8) between a first portion and a second portion of the central member 112. Similarly, the second bend 136 may form a second angle (a) between the second portion and a third portion of the central member 112. The first angle (8) and the second angle (a) may be different angles in some embodiments. For instance, in some embodiments, the first angle (8) may be approximately 45 degrees while the second angle (a) may be approximately 90 degrees.

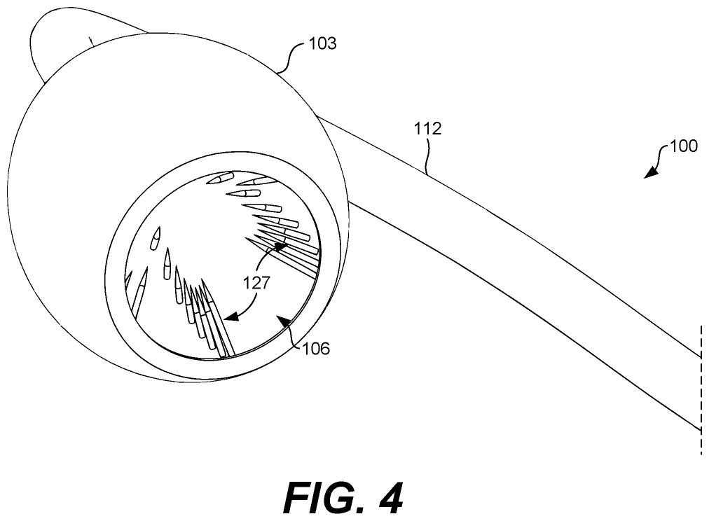

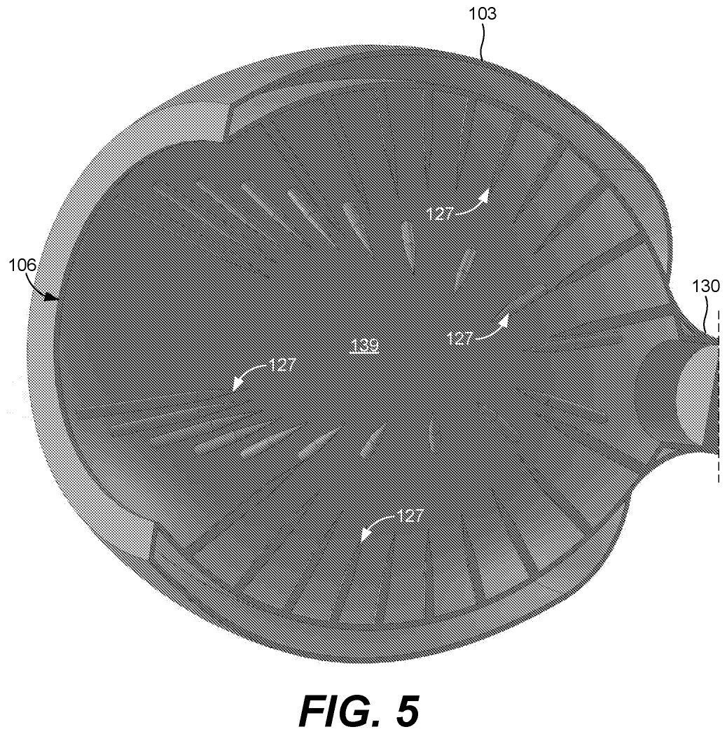

Referring to FIG. 5, a cross-section of the needle head 103 is shown according to various embodiments. The cavity 106 may be described as an aperture and a hollow chamber of the needle head 103. The aperture of the needle head 103 may be substantially similar to a size of a tonsil of one of: an adult male, adult female, adolescent male, adolescent female, or an animal. The needle array 127 may be disposed on an interior surface 139 of the needle head 103. As may be appreciated, the arrangement of the needles in the needle array 127 may vary based on a desired application. For instance, the arrangement of the needles (e.g., a number of the needles and the placement of the needles) may vary based on a compound or a type of tissue being treated. As can be seen in FIGS. 4 and 5, an interior surface of the cavity 106 is elliptically concave, thus the needles in the needle array 127 point towards a center of the needle head 103. In some embodiments, the needle array 127, as opposed to the needle head 103 itself, may be detachably attached from the medical instrument 100, thereby leaving the needle array 127 in the tissue for an extended period of time.

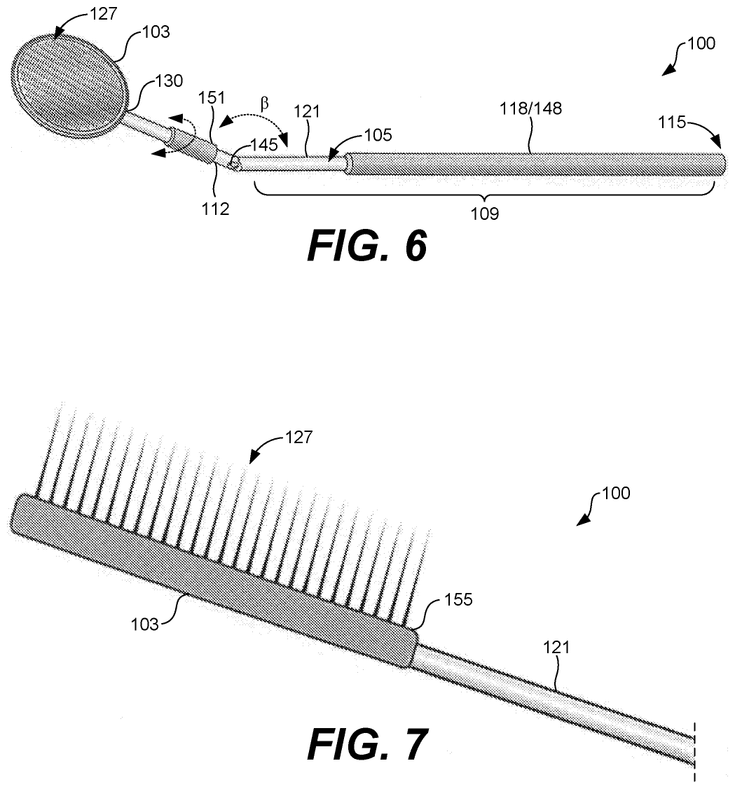

Turning now to FIG. 6, another embodiment of the medical instrument 100 is shown according to various embodiments. In the non-limiting example of FIG. 6, an angle (1) between the handle portion 109 and the central member 112 may be adjusted using a pivot screw 145 or other pivot means. Further, the first elongated member 118 of the handle portion 109 may include a handle 148 for gripping or otherwise holding the medical instrument 100. Additionally, the second elongated member 121 may include an elongated rod that is circular-shaped, where at least a portion of the elongated rod is received in a recess of the handle 148. The handle portion 109 may be coupled to the central member 112 that is also circular-shaped at a distal end of the second elongated member 121 by way of the pivot screw 145. To this end, the handle portion 109 is pivotably coupled to the central member 112, where the curvature of the medical instrument 100 is formed and adjustable based on an angle of pivot formed between the handle portion 109 and the central member 112. In further embodiments, the pivot screw 145 may include a bearing, a nut, a pivot rod, or other appropriate coupling mechanism to adjust the angle of pivot, as may be appreciated.

Additionally, in some embodiments, at least a portion of the central member 112 may be disposed in a rotating sleeve 151. As may be appreciated, a manual rotation of the rotating sleeve 151 by an operator may cause a rotation of the portion of the central member 112, the needle head 103, and the needle array 127. While the embodiment of FIG. 6 shows the needle head 103 as being flat and circular, in other embodiments, the needle head 103 may be square- or rectangular-shaped. Further, the needle head 103 shown in FIG. 1 may be employed with the central member 112 and the handle portion 109 of FIG. 6.

As noted above, a suction of tissue, saliva, or other content may be applied in association with the medical instrument 100. For example, suction may be employed to assist with securing the needle head 103 to the tissue. In some embodiments, an external suction instrument may be used where a tube or other apparatus is coupled to the terminal end 115 of the medical instrument 100. For instance, in some embodiments, a passageway may be formed throughout the handle portion 109, the central member 112, and the needle head 103 where an external suction instrument may be detachably attached to the terminal end 115 of the handle portion 109. As a result, air may be pulled through the passageway or a fluid compound may be pumped towards the needle array 127 for introducing into tissue of a patient. A hole or outlet (not shown) for the passageway can be introduced at a suitable location of the needle head 103, such as a center of the needle head 103, to enable suction that secures the needle head 103 to the tissue during a delivery of the compound.

Referring next to FIG. 7, an enhanced cross-section of the needle head 103 of the medical instrument 100 is shown according to various embodiments of the present disclosure. In the embodiment of FIG. 7, the needles in the needle array 127 are positioned on a needle head surface 155 in a uniform arrangement where each of the needles has a uniform length, thus forming a substantially flat needle surface arrangement.

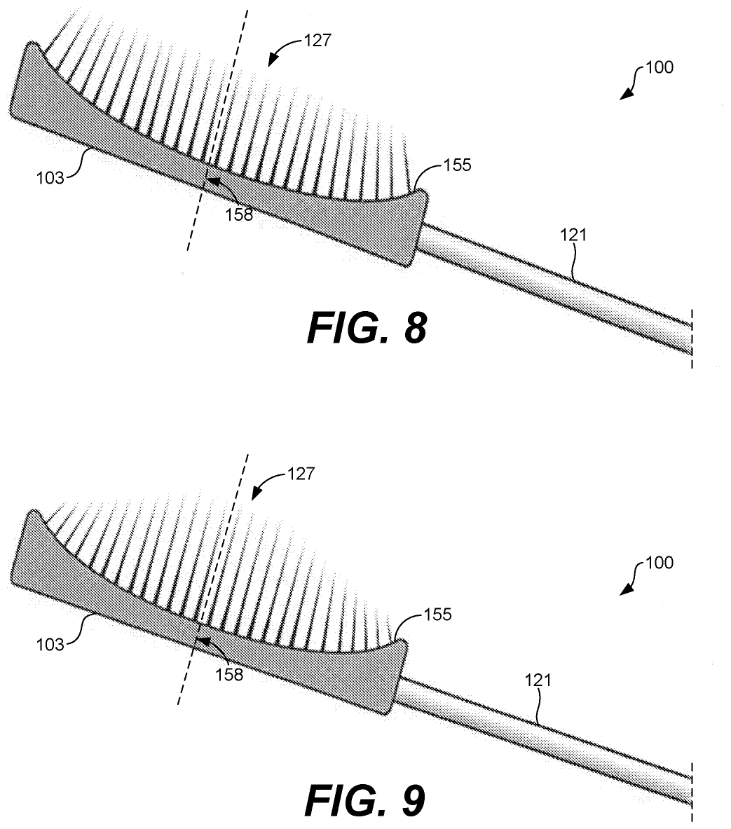

Turning now to FIG. 8, another enhanced cross-section of the needle head 103 of the medical instrument 100 is shown according to another embodiment of the present disclosure. In the non-limiting example of FIG. 8, the surface 155 of the needle head 103 may include a recessed and inwardly curved surface. As the needle array 127 is disposed on the recessed surface 155 of the needle head 103, at least a portion of the needles in the needle array 127 point inwards towards a center 158 of the needle head 103. The shape of the needle array 127 may provide better insertion into the epithelial surface of the tonsil.

Moving on to FIG. 9, another enhanced cross-section of the needle head 103 of the medical instrument 100 is shown according to another embodiment of the present disclosure. Similar to the embodiment described in FIG. 8, in the non-limiting example of FIG. 9, the surface 155 of the needle head 103 may be recessed. As the needle array 127 is disposed on the recessed surface 155 of the needle head 103, at least a portion of the needles in the needle array 127 point inwards towards a center 158 of the needle head 103, similar to the embodiment of FIG. 8. However, the needle array 127 shown in FIG. 9 includes needles having a length varying from other ones of the needles. For instance, needles at the edges of the needle head 103 may be shorter than needles located in the interior of the needle head 103, which can become increasing long. Thus, the needles in the needle array 127 may project outwards, thereby forming an outwardly-facing curve or a bell-shaped curve. The needles in the center of the needle array 127 may have the longest length to deliver drugs or other compounds efficiently into the core of the tonsil. In some embodiments, the needle head surface 155 is elliptically concave.

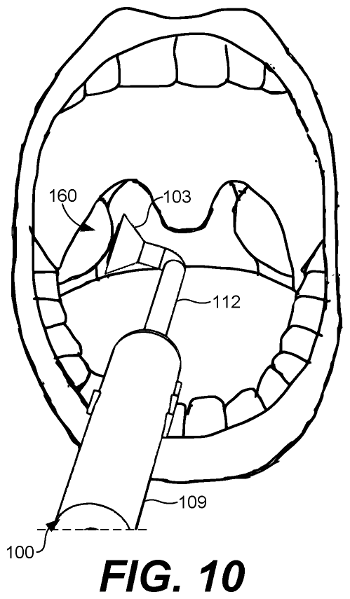

Moving on to FIG. 10, the medical instrument 100 is shown introducing tonsillar tissue 160 of a patient into the needle head 103. As may be appreciated, the medical instrument 100 described herein is adapted for accessing tonsillar tissue 160 other appropriate region of the oral or pharyngeal cavity. The needle head 103 includes a multitude of needles or microneedles disposed in a cavity 106 therein for introducing a compound into the tonsillar tissue 160. in some embodiments, after the tonsillar tissue 160 is introduced into the needle head 103, the needle head 103 or the needle array 127 may be detached from the central member 112, leaving the needle head 103 and/or the needle array 127 attached to the tonsillar tissue of the patient.

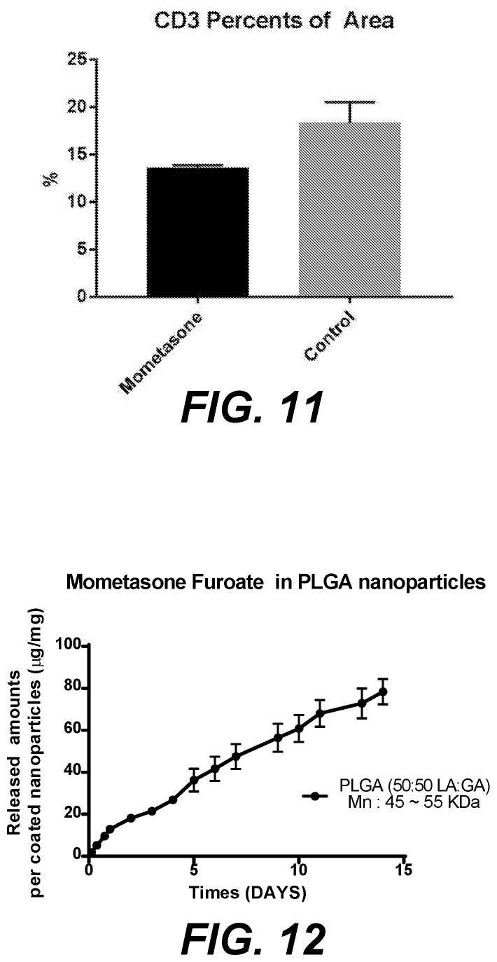

Referring now to FIG. 11, a graph is shown illustrating a percentage of cluster-of-differentiation-3 (CD3) T-cell co-receptors in a rabbit when treated with mometasone versus a control. In immunology, the CD3 T-cell co-receptor helps to activate both the cytotoxic T-Cell (CD8+ naive T cells) and also T helper cells (CD4+ naive T cells). CD3 consists of a protein complex and is composed of four distinct chains. As shown in FIG. 11, the CD3 positive T-cells were observed being significantly lower in rabbit tonsils injected with mometasone (steroid), which may represent the T-cell apoptosis (natural cell death) in the tonsils.

Turning now to FIG. 12, a chart is shown illustrating in vitro kinetics of a drug or compound released into tonsillar tissue. For instance, a mometasone furoate diffusion profile was used from 50:50 (Lactide:Glycolide) PLGA [Poly(Lactide-co-Glycolide)] nanoparticles in a 50% ethanol aqueous solution. The values represent the mean±standard deviation of three or four batches. The released amount per coated nanoparticles is shown versus time.

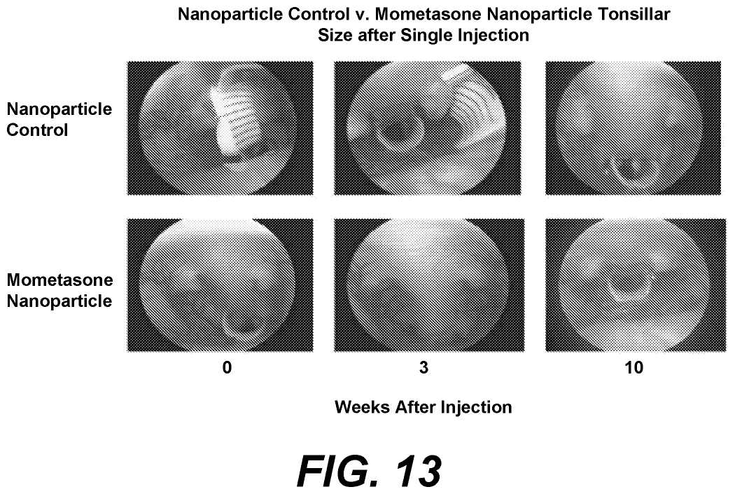

Referring next to FIG. 13, photographs of tonsillar tissue are shown, where the first row included photographs of tonsillar tissue treated with a nanoparticle control and the second row includes photographs of tonsillar tissue treated with a mometasone nanoparticle. Both were performed using a sustained release of mometasone nanoparticles delivered by a needle head 103 (and needle array 127) that can be detached from the central member 112 and left in the tonsillar tissue for a period of weeks. In experiments performed in accordance with the embodiments described herein, a total of 16 tonsils (mometasone-nanoparticle:nanoparticle alone=8:8) were analyzed. The tonsils that received the mometasone-nanoparticles were observed as being significantly smaller than the nanoparticle alone-injected tonsils at week three and week ten, where p<0.05.

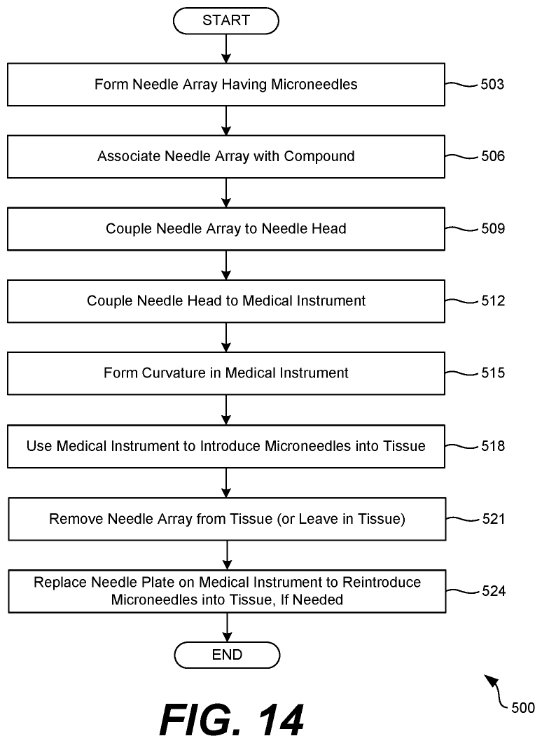

Turning now to FIG. 14, a flowchart 500 is shown illustrating an example method for using the medical instrument 100 described herein or otherwise introducing a compound into tissue according to various embodiments of the present disclosure. Starting with step 503, a needle array 127 may be formed having an arrangement of needles or microneedles. Next, in step 506, the needle array 127 may be associated with a compound.

In some embodiments, the needle array 127 is associated with the compound by virtue of the needles being formed of the compound. For instance, the needles in the needle array 127 may be formed of a steroid or other compound (or combination of other compounds) during manufacture and be configured such that the needles dissolve when introduced into tonsillar or other tissue. A rate of dissolution may vary based on the compounds, as may be appreciated. Alternatively, the needles of the needle array 127 may be dipped into, coated with, or otherwise associated with the compound. In further embodiments, the needle head 103 or other portion of the medical instrument 100 may include a hollow interior having a reservoir where the compound is stored for pumping into the needles of the needle array 127. To this end, the needle array 127 may be coupled to the needle head 103 such as to permit the compound to be delivered into tissue through the needles of the needle array 127.

In step 509, the needle array 127 may be coupled to the needle head 103 and, in step 512, the needle head 103 may be coupled to the medical instrument 100. For instance, the needle head 103 may be detachably attached to the distal end of the central member 112. It is understood that the needle head 103 may be removed from the distal end of the central member 112 via a release button 124 or similar mechanism.

Next, in step 515, a curvature may be formed in the medical instrument 100 if not present. For example, an angle of pivot between the handle portion 109 and the central member 112 may be adjusted to an angle suitable for reaching the structure in the oral or pharyngeal cavity. Next, in step 518, the medical instrument 100 may be used by an operator to navigate a cavity and introduce the needle array 127 into tissue, such as tonsillar tissue. As may be appreciated, the compound may be delivered as the needle array 127 is punctured or otherwise introduced into the tissue.

In step 521, the needle array 127 may be removed from the tissue, for example, after needles of the needle array 127 have dissolved or the compound has otherwise been sufficiently introduced into the tissue. Next, in step 524, the needle head 103 may be replaced with another needle head 103 to reintroduce the compound into tissue of the subject at a later time, if needed.

Embodiments of the present disclosure provide a tonsil-accessible medical instrument 100 having a detachably attached needle head 103 which may be used as an alternative to T&A. The medical instrument 100 may deliver therapeutic drugs, including steroids, in which needles or microneedles can deliver compounds in the surface and core of the tonsil. In further embodiments of the disclosure, in order to prevent significant loss of drugs due to saliva, the needle head 103 may be inserted into and maintained within the tonsillar tissue for a suitable period of time to confer a long-term drug delivery into the tissue.

Using dissolving needles or microneedles holding a drug or a combination of drugs, a systemic and sustainable intratonsillar drug delivery may be achieved. In further embodiments, dissolution of each microneedle may be adjustable. For example, the needles or microneedles may be formed of biocompatible materials (such as carboxymethylcellulose or hyaluronic acid) that have different dissolution kinetics after inserting into the tissue. Drug-loaded micro particles may be incorporated into the needles or microneedles in the needle array 127 or coated onto the tip (or “arrowhead”) of the needles, or, in other embodiments, introduced into a backing material or reservoir of the needles.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The potential applications of the present invention are not limited to tissue engineering. Other areas of application may include cosmetics, veterinarian treatment, pharmaceutical applications, and retail, among others. It should be emphasized that the above-described embodiments of the present disclosure are merely possible examples of implementations set forth for a clear understanding of the principles of the disclosure. Many variations and modifications may be made to the above-described embodiments without departing substantially from the spirit and principles of the disclosure. All such modifications and variations are intended to be included within the scope of this disclosure and protected by the following claims.

Clause 1: A medical instrument, comprising: a handle portion; a central member coupled to a distal end of the handle portion; and a needle head coupled to a distal end of the central member, the needle head comprising a needle array disposed in a cavity of the needle head, wherein the needle array comprises a plurality of needles configured with a compound that, when the plurality of needles are introduced into tissue, causes a delivery of the compound into the tissue.

Clause 2: The medical instrument of clause Error! Bookmark not defined., wherein the plurality of needles are a plurality of microneedles, each of the plurality of microneedles having a needle size of about 250 micrometers to 2,500 micrometers.

Clause 3: The medical instrument of clauses 1-2, wherein the needle head comprises an ovular cylinder.

Clause 4: The medical instrument of clauses 1-3, wherein the needle head comprises an elliptically concave inner surface, wherein each of the plurality of microneedles are disposed along the elliptically concave inner surface.

Clause 5: The medical instrument of clauses 1-4, wherein the central member is C-shaped, the central member comprising a first bend forming a first angle between a first portion of the central member and a second portion of the central member, and a second bend forming a second angle between the second portion of the central member and a third portion of the central member.

Clause 6: The medical instrument of clauses 1-5, wherein at least one of the plurality of microneedles in the needle array has a length varying from other ones of the plurality of microneedles.

Clause 7: The medical instrument of clauses 1-6, wherein the compound is at least one of: a steroid, an antibiotic, or a therapeutic compound.

Clause 8: The medical instrument of clauses 1-7, wherein the tissue is tonsillar tissue selected from at least one of: palatine tonsils, pharyngeal tonsil (adenoid), and lingual tonsils.

Clause 9: The medical instrument of clauses 1-9, wherein each of the plurality of microneedles are configured to dissolve into the tissue to introduce the compound.

Clause 10: The medical instrument of clauses 1-9, wherein at least one of the needle head and the needle array is detachably attachable from the central member, wherein the medical instrument comprises at least one button configured to release the needle head or the needle array from the central member.

Clause 11: A method for intratonsillar drug delivery, comprising: providing a medical instrument, the medical instrument comprising: a handle portion; a central member coupled to a distal end of the handle portion; and a needle head coupled to a distal end of the central member, the needle head comprising a needle array disposed in a cavity of the needle head; associating the needle array with a compound, wherein the needle array comprises a plurality of needles; and introducing the plurality of needles in the needle array into tonsillar tissue, thereby causing a delivery of the compound into a tonsil of a subject.

Clause 12: The method of clause 11, wherein associating the needle array with the compound comprises at least one of: forming the plurality of needles using the compound; dipping the plurality of needles into the compound; or introducing the compound into through a reservoir maintained in the medical instrument, wherein the needle array is coupled to the needle head permitting the compound to be delivered into the tonsillar tissue through the plurality of needles.

Clause 13: The method of clauses 11-12, wherein each of the plurality of needles are a microneedle having a needle size of about 250 micrometers to 2,500 micrometers.

Clause 14: The method of clauses 11-13, wherein introducing the needle array into tonsillar tissue causes the plurality of needles to dissolve into the tonsillar tissue.

Clause 15: The method of clauses 11-14, further comprising coupling the needle array to the needle head of the medical instrument by: coupling the needle array to the cavity of the needle head; and coupling the needle head to a distal end of the medical instrument.

Clause 16: The method of clauses 11-15, further comprising applying a suction in association with the medical instrument.

Clause 17: The method of clauses 11-16, wherein the compound is at least one of: a steroid, an antibiotic, or a therapeutic compound.

Clause 18: The method of clauses 11-17, wherein at least one of the needle head and the needle array is detachably attachable from the central member, wherein the method further comprises releasing the needle head or the needle array using a button on the medical instrument, wherein the needle head or the needle array is maintained in the tonsillar tissue when released.

Clause 19: The method of clauses 11-18, wherein the method of claim 11 is applied to treat tonsillar hypertrophy or an inflammation of the tonsil.

Clause 20: The method of clauses 11-19, wherein the central member is C-shaped, the central member comprising a first bend forming a first angle between a first portion of the central member and a second portion of the central member, and a second bend forming a second angle between the second portion of the central member and a third portion of the central member.

Therefore, the following is claimed:

1. A medical instrument, comprising:
a handle portion;
a central member coupled to a distal end of the handle portion; and
a needle head coupled to a distal end of the central member, the needle head comprising a needle array disposed in a cavity of the needle head, wherein the needle array comprises a plurality of needles configured with a compound that, when the plurality of needles are introduced into tissue, causes a delivery of the compound into the tissue.

2. The medical instrument of claim 1, wherein the plurality of needles are a plurality of microneedles, each of the plurality of microneedles having a needle size of about 250 micrometers to 2,500 micrometers.

3. The medical instrument of claim 2, wherein the needle head comprises an ovular cylinder.

4. The medical instrument of claim 3, wherein the needle head comprises an elliptically concave inner surface, wherein each of the plurality of microneedles are disposed along the elliptically concave inner surface.

5. The medical instrument of claim 1, wherein the central member is C-shaped, the central member comprising a first bend forming a first angle between a first portion of the central member and a second portion of the central member, and a second bend forming a second angle between the second portion of the central member and a third portion of the central member.

6. The medical instrument of claim 2, wherein at least one of the plurality of microneedles in the needle array has a length varying from other ones of the plurality of microneedles.

7. The medical instrument of claim 1, wherein the compound is at least one of: a steroid, an antibiotic, or a therapeutic compound.

8. The medical instrument of claim 1, wherein the tissue is tonsillar tissue selected from at least one of: palatine tonsils, pharyngeal tonsil (adenoid), and lingual tonsils.

9. The medical instrument of claim 2, wherein each of the plurality of microneedles are configured to dissolve into the tissue to introduce the compound.

10. The medical instrument of claim 1, wherein at least one of the needle head and the needle array is detachably attachable from the central member, wherein the medical instrument comprises at least one button configured to release the needle head or the needle array from the central member.

11. A method for intratonsillar drug delivery, comprising:
providing a medical instrument, the medical instrument comprising:
a handle portion;
a central member coupled to a distal end of the handle portion; and
a needle head coupled to a distal end of the central member, the needle head comprising a needle array disposed in a cavity of the needle head;
associating the needle array with a compound, wherein the needle array comprises a plurality of needles; and
introducing the plurality of needles in the needle array into tonsillar tissue, thereby causing a delivery of the compound into a tonsil of a subject.

12. The method of claim 11, wherein associating the needle array with the compound comprises at least one of:
forming the plurality of needles using the compound;
dipping the plurality of needles into the compound; or
introducing the compound into through a reservoir maintained in the medical instrument, wherein the needle array is coupled to the needle head permitting the compound to be delivered into the tonsillar tissue through the plurality of needles.

13. The method of claim 12, wherein each of the plurality of needles are a microneedle having a needle size of about 250 micrometers to 2,500 micrometers.

14. The method of claim 11, wherein introducing the needle array into tonsillar tissue causes the plurality of needles to dissolve into the tonsillar tissue.

15. The method of claim 11, further comprising coupling the needle array to the needle head of the medical instrument by:
coupling the needle array to the cavity of the needle head; and
coupling the needle head to a distal end of the medical instrument.

16. The method of claim 11, further comprising applying a suction in association with the medical instrument.

17. The method of claim 11, wherein the compound is at least one of: a steroid, an antibiotic, or a therapeutic compound.

18. The method of claim 11, wherein at least one of the needle head and the needle array is detachably attachable from the central member, wherein the method further comprises releasing the needle head or the needle array using a button on the medical instrument, wherein the needle head or the needle array is maintained in the tonsillar tissue when released.

19. The method of claim 11, wherein the method of claim 11 is applied to treat tonsillar hypertrophy or an inflammation of the tonsil.

20. The method of claim 11, wherein the central member is C-shaped, the central member comprising a first bend forming a first angle between a first portion of the central member and a second portion of the central member, and a second bend forming a second angle between the second portion of the central member and a third portion of the central member.
